(19) United States
(12) Patent Application Publication
(10) Pub. No.: US 2009/0124643 A1
(43) Pub. Date: May 14, 2009

(54) Use of a Cannabinoid CB1 Receptor Antagonist for Preparation of Drugs Useful for the Prevention and Treatment of Benign Prostatic Hypertrophy

(75) Inventors: Tiziano Croci, Milan (IT); Fabio Guagnini, Milano (IT); Roberta Avallone, Romano di Lombardia (Bergamo) (IT)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/327,298

(22) Filed: Dec. 3, 2008

Related U.S. Application Data
(63) Continuation of application No. PCT/FR2007/000913, filed on Jun. 1, 2007.

(30) Foreign Application Priority Data
Jun. 7, 2006 (FR) 0605074

(51) Int. Cl. A61K 31/517; A61K 31/454; A61P 13/08

(52) U.S. Cl. 514/266.24; 514/326

(57) ABSTRACT

The present invention relates to the use of cannabinoid CB1 receptor antagonist for the preparation of drugs useful for the prevention and treatment of benign prostatic hypertrophy.

This application is a continuation of International application No. PCT/FR2007/000,913, filed Jun. 1, 2007, which is incorporated herein by reference in its entirety; which claims the benefit of priority of French Patent Application No. 06/05,074 filed Jun. 7, 2006.

The present invention relates to the use of a cannabinoid CB1 receptor antagonist for the preparation of medicaments for use in the prevention and treatment of benign prostatic hypertrophy.

More particularly, the present invention relates to the use of a cannabinoid CB1 receptor antagonist derived from pyrazole.

According to the present invention, the term “cannabinoid CB1 receptor antagonist derived from pyrazole” is intended to mean a compound chosen from N-piperidino-5-(4-chlorophenyl)-1-(2,4-dichlorophenyl)-4-methylpyrazole-3-carboxamide, the international non-proprietary name of which is rimonabant, described in European patent EP 656 354, and N-piperidino-5-(4-bromophenyl)-1-(2,4-dichlorophenyl)-4-ethylpyrazole-3-carboxamide, the international nonproprietary name of which is surinabant, described in European patent EP 1 150 961.

Benign prostatic hypertrophy (BPH) is a condition which is defined anatomically by an increase in prostate size not due to a cancer, and histologically by fibromuscular and glandular hyperplasia.

BPH is quite a common condition and can occur in men from the age of 40 onward. The increase in prostate size can cause partial or total obstruction of the urethra, which is reflected by a decrease in the flow of seminal fluid and urine.

The possible use of cannabinoid receptor modulators for treating prostate cancers is described in many patent applications (for example: WO 2001/087 297, WO 2003/086 288, WO 2005/067 917). Furthermore, results in the literature (S. Sarfaraz et al. Cancer Res. 2005, 65(5), 1635-1641) show the effects of WIN-55,212-2, a mixed CB₁/CB₂ agonist, on the proliferation and aptosis of cancerous prostate cells.

It has now been found that cannabinoid CB1 receptor antagonist compounds are active in decreasing the size of a noncancerous hypertrophied prostate and, consequently, in preventing or treating benign prostatic hypertrophy.

Thus, according to the present invention, a cannabinoid CB1 receptor antagonist compound, in particular a pyrazole-derived cannabinoid receptor antagonist, chosen from rimonabant and N-piperidino-5-(4-bromophenyl)-1-(2,4-dichlorophenyl)-4-ethylpyrazole-3-carboxamide, can be used for the preparation of medicaments for use in preventing and treating benign prostatic hypertrophy.

The pharmaceutical compositions according to the present invention contain an effective dose of a cannabinoid CB1 receptor antagonist compound, in particular a pyrazole-derived cannabinoid receptor antagonist compound, chosen from rimonabant and surinabant, and also at least one pharmaceutically acceptable excipient.

Said excipients are chosen, according to the pharmaceutical form and the method of administration desired, from the usual excipients which are known to those skilled in the art.

In the pharmaceutical compositions of the present invention for oral, sublingual, subcutaneous, intramuscular, intravenous, topical, local, intratracheal, intranasal, transdermal or rectal administration, the active ingredient may be administered in unit administration form, as a mixture with conventional pharmaceutical excipients, to animals and to humans for the prevention or treatment of the disorders and diseases above.

Suitable unit administration forms comprise oral administration forms such as tablets, soft or hard gel capsules, powders, granules and oral solutions or suspensions, sublingual, buccal, intratracheal, intraocular of intranasal administration forms, forms for administration by inhalation, topical, transdermal, subcutaneous, intramuscular or intravenous administration forms, rectal administration forms, and implants. For topical application, the compounds according to the invention may be used in creams, gels, ointments or lotions.

Oral administration forms such as gel capsules or tablets are preferred.

More particularly, gel capsules or tablets containing rimonabant at a dose of between 5 and 50 mg, more particularly the doses of 5, 10 and 20 mg, are preferred.

Furthermore, for the use according to the present invention, a pyrazole-derived cannabinoid receptor antagonist, chosen from rimonabant and surinabant, may be combined with another active ingredient that is of use in the treatment of BPH, for example an alpha-blocker such as alfuzosin, tamsulosin, terazosin, doxazosin, prazosin or indoramine, or else a 5-alpha-reductase inhibitor such as finasteride or dutasteride.

According to another particular embodiment, a subject of the present invention is the use of a pharmaceutical composition containing a combination of rimonabant and alfuzosin, of use for the prevention and treatment of benign prostatic hypertrophy.

A subject of the invention is also the use of a composition containing a combination of surinabant and alfuzosin, of use for the prevention and treatment of benign prostatic hypertrophy.

According to another aspect of the invention, the pyrazole-derived cannabinoid receptor antagonist, chosen from rimonabant and surinabant, and the other active ingredient that is combined, may be administered simultaneously, separately or sequentially.

“Separate use” is intended to mean the administration, at the same time, of the two compounds of the composition according to the invention, each in a distinct pharmaceutical form.

“Sequential use” is intended to mean the successive administration of the first compound of the composition according to the invention, in one pharmaceutical form, and then of the second compound of the composition according to the invention, in a separate pharmaceutical form.

In the case of this “sequential use”, the time elapsed between the administration of the first compound of the composition according to the invention and the administration of the second compound of the same composition according to the invention does not generally exceed 24 hours; it may be longer if one or other of the compounds is in a pharmaceutical formulation which allows, for example, a weekly administration.

The pharmaceutical forms, comprising either just one of the compounds making up the composition according to the invention or the combination of the two compounds, or where appropriate, of three compounds, which can be employed in the various types of uses described above, can, for example, be suitable for oral, nasal, parenteral or transdermal administration.

Also, in the case of a “separate use” and of a “sequential use”, two distinct pharmaceutical forms may be intended for the same route of administration or for a different route of administration (oral and transdermal, or oral and nasal, or parenteral and transdermal, etc.).

### EXAMPLE 1

### Treatment of Testosterone-Induced BPH in Rats

Male rats are implanted with catheters and receive 18.75 mg per rat of testosterone, subcutaneously at D-4.

From day D0 to day D21, they receive rimonabant daily, at the dose of either 10 mg/kg per os, or of 30 mg/kg per os.

At day D22, the animals are sacrificed and their prostate is analyzed.

The pretreatment with testosterone increased the weight and the volume of the total prostate and of the ventral prostate of the rats by approximately 35%.

In the rats receiving the chronic treatment with rimonabant, a significant decrease in weight and volume of the prostate is observed.

In parallel, a decrease in weight and volume of the ventral prostate is observed for the animals treated with rimonabant:

Rimonabant

| | 10 mg/kg/d | 30 mg/kg/d |
|---|---|---|
| Decrease in weight of | 54% | 73% |
| total prostate | | |
| Decrease in volume of | 64% | 87% |
| total prostate | | |
| Decrease in weight of | 87% | 95% |
| ventral prostate | | |
| Decrease in volume of | 88% | 100% |
| ventral prostate | | |

This decrease is evaluated as % of the increase preinduced with testosterone.

### EXAMPLE 2

### Treatment of BPH in Spontaneously Hypertensive Rats

The spontaneously hypertensive rat (SHR) model as described by Golomb et al., J. Androl. (2000), 21(1), 58-64 and Matituahou et al., J. Androl. (2003) 24(2), 263-9 is used.

A qualitative histological study of the prostate lobes is carried out on untreated SHR rats and on rats treated for 28 days with 10 mg/kg or 30 mg/kg of rimonabant. In the untreated rats, the lobes of the ventral prostate show multiple epithelial layers and a hypertrophic stroma; whereas, in the treated rats, from 10 mg/kg onward, the tissues are normal and non-hypertrophied.

What is claimed is:

1. A method of treatment of benign prostatic hypertrophy comprising administering to a patient in need of said treatment a therapeutically effective amount of pyrazole-derived cannabinoid CB1 receptor antagonist compound.

2. The method, as claimed in claim 1, wherein the pyrazole-derived cannabinoid CB1 receptor antagonist is combined with a pharmaceutically acceptable excipient.

3. The method, as claimed in claim 1, wherein the pyrazole-derived cannabinoid CB1 receptor antagonist is chosen from rimonabant and surinabant.

4. The method, as claimed in claim 1, wherein the pyrazole-derived cannabinoid CB1 receptor antagonist is rimonabant.

5. The method, as claimed in claim 1, wherein the pyrazole-derived cannabinoid CB1 receptor antagonist is surinabant.

6. The method, as claimed in claim 1, wherein the pyrazole-derived cannabinoid CB1 receptor antagonist is combined with another active ingredient suitable for the treatment of benign prostatic hypertrophy.

7. The method, as claimed in claim 2, wherein the pyrazole-derived cannabinoid CB1 receptor antagonist is combined with another active ingredient suitable for the treatment of benign prostatic hypertrophy.

8. The method, as claimed in claim 3, wherein rimonabant or surinabant is combined with another active ingredient suitable for the treatment of benign prostatic hypertrophy.

9. The method, as claimed in claim 4, wherein rimonabant is combined with another active ingredient suitable for the treatment of benign prostatic hypertrophy.

10. The method, as claimed in claim 5, wherein surinabant is combined with another active ingredient suitable for the treatment of benign prostatic hypertrophy.

11. The method, as claimed in claim 9, wherein rimonabant is combined with alfuzosin.

12. The method, as claimed in claim 10, wherein surinabant is combined with alfuzosin.
